(19) United States
(12) Patent Application Publication
(10) Pub. No.: US 2022/0199242 A1
(43) Pub. Date: Jun. 23, 2022

(54) STORAGE MANAGEMENT DEVICE FOR MEDICAL INSTRUMENT, STORAGE MANAGEMENT SYSTEM, AND STORAGE MANAGEMENT METHOD FOR MEDICAL INSTRUMENT

(71) Applicant: DGSHAPE Corporation, Hamamatsu-shi (JP)

(72) Inventors: Akinori SUGAYA, Hamamatsu-shi (JP); Masaki HANAJIMA, Hamamatsu-shi (JP); Akira OTAKA, Hamamatsu-shi (JP); Takaaki KOKUBO, Hamamatsu-shi (JP)

(21) Appl. No.: 17/558,668

(22) Filed: Dec. 22, 2021

(30) Foreign Application Priority Data
Dec. 23, 2020 (JP) 2020-214045

(51) Int. Cl. G16H 40/40; A61L 2/26; A61L 2/24

(52) U.S. Cl. CPC G16H 40/40; A61L 2202/24; A61L 2/24; A61L 2/26

(57) ABSTRACT

A memory of a storage management device stores storage history information in which at least a storage ID of an instrument set including one or more medical instruments is stored and a sterilization ID indicating that sterilization processing has been performed on the instrument set are associated with one another. A processor executes sterilization ID acquisition processing to acquire a target sterilization ID of an instrument set that is a target to be stocked, determination processing to determine whether the sterilization ID that matches the target sterilization ID is included in the storage history information, and notification processing to notify, when it is determined in the determination processing that the sterilization ID that matches the target sterilization ID is included in the storage history information, that the instrument set having the target sterilization ID has been re-stocked.

### CROSS REFERENCE TO RELATED APPLICATIONS

This application claims the benefit of priority to Japanese Patent Application No. 2020-214045, filed on Dec. 23, 2020. The entire contents of this application are incorporated herein by reference.

### BACKGROUND OF THE INVENTION

### 1. Field of the Invention

The present invention relates to a storage management device for a medical instrument, a storage management system, and a storage management method for a medical instrument.

### 2. Description of the Related Art

For example, Japanese Laid-open Patent Publication No. 2017-148113 discloses a medical instrument repeatedly used in a medical practice, such as a surgical operation, a medical examination, or the like. The medical instrument can be repeatedly used by sequentially performing predetermined medical treatment processes. The medical treatment processes include a surgical operation process in which a surgical operation is performed, a collection process, a washing process, an assembling process, a sterilization process, and a storage process.

In the collection process, a medical instrument is collected after a surgical operation. In the washing process, the collected medical instrument is disassembled into a plurality of separated components and the components are washed. In the assembling process, the components of the medical instrument that have been washed are assembled. In the sterilization process, sterilization processing is performed on the medical instrument that has been assembled. In the storage process, the sterilized medical instrument is stored in a predetermined storage area. In the surgical operation process, the medical instrument stored in a storage area is dispensed and a surgical operation is performed using the dispensed medical instrument.

Incidentally, when a surgical operation is performed in the surgical operation process, a predetermined medical instrument that is planned to be used in a surgical operation is dispensed. However, among dispensed medical instruments, there can be medical instruments that are not actually used. The medical instruments that have not been actually used in the surgical operation are stored again in the storage process without going through the washing process, the assembling process, the sterilization process, or the like. Being stored again without being used in a surgical operation and also without going through sterilization will be hereinafter referred to as being re-stocked.

The above-described re-stocking of medical instruments has not been strictly managed. Depending on a medical site, re-stocking of medical instruments is managed, for example, using a paper-based system. Re-stocked medical instruments have not been essentially needed to be dispensed from a storage area in which the medical instruments are stored. As the number of re-stocked medical instruments increases, the number of works performed for dispensing the medical instruments from the storage area where the medical instruments are stored increases, and a dispensing work becomes insufficient. Therefore, it is preferable that the number of medical instruments that are re-stocked is reduced.

### SUMMARY OF THE INVENTION

Preferred embodiments the present invention provide storage management devices for medical instruments, storage management systems, and storage management methods for medical instruments by which re-stocked medical instruments can be easily known.

A storage management device for a medical instrument according to a preferred embodiment of the present invention includes a memory and at least one processor. The storage management device stores storage history information in which at least a storage ID that is assigned to an instrument set including one or more medical instruments when the instrument set is stored in the memory and a sterilization ID indicating that sterilization processing has been performed on the instrument set are associated with one another. The at least one processor is configured or programmed to execute a program stored in the memory to perform sterilization ID acquisition processing, determination processing, and notification processing. In the sterilization ID acquisition processing, a target sterilization ID assigned to an instrument set that is a target to be stocked is acquired. In the determination processing, whether the sterilization ID that matches the target sterilization ID is included in the storage history information is determined. In the notification processing, when it is determined in the determination processing that the sterilization ID that matches the target sterilization ID is included in the storage history information, it is notified that the instrument set assigned the target sterilization ID has been re-stocked.

According to a storage management device for a medical instrument according to a preferred embodiment the present invention, the target sterilization ID is an ID that is assigned when sterilization processing is performed on the instrument set. The sterilization processing is performed on the instrument set that has been used in a surgical operation, a medical examination, or the like and, even to the same instrument set, each time sterilization processing is performed, a new target sterilization ID is assigned. Herein, if there is the sterilization ID that matches the target sterilization ID acquired in the sterilization ID acquisition processing in the storage history information, it is meant that the instrument set that has been dispensed once is stocked again without being used in a surgical operation or a medical examination and without going through the sterilization processing. Accordingly, when it is determined that the sterilization ID that matches the target sterilization ID is included in the storage history information, it can be determined that the instrument set assigned the target sterilization ID has been re-stocked. If the instrument set has been re-stocked, re-stocking is notified in the notification processing, so that an operator can be easily know that the instrument set has been re-stocked.

In a storage management method for a medical instrument according to a preferred embodiment of the present invention, storage history information in which at least a storage ID that is assigned to an instrument set including one or more medical instruments when the instrument set is stored and a sterilization ID indicating that sterilization processing has been performed on the instrument set are associated with one another is stored. The storage management method includes a sterilization ID acquisition step, a determination step, and a notification step. In the sterilization ID acquisition step, a target sterilization ID assigned to an instrument set that is a target to be stocked is acquired. In the determination step, whether the sterilization ID that matches the target sterilization ID is included in the storage history information is determined. In the notification step, when it is determined in the determination step that the sterilization ID that matches the target sterilization ID is included in the storage history information, it is notified that the instrument set assigned the target sterilization ID has been re-stocked.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic view illustrating a storage management system according to a preferred embodiment of the present invention.

FIG. 2 is a diagram illustrating a circulation cycle.

FIG. 3 is a block diagram of a storage management device.

FIG. 4 is a table illustrating a configuration example of an instrument set master table.

FIG. 5 is a table illustrating a configuration example of a rack master table.

FIG. 6 is a table illustrating a configuration example of a sterilization table.

FIG. 7 is a table illustrating a configuration example of a storage history table.

FIG. 8 is a flowchart illustrating process steps of processing of the storage management device in stocking an instrument set.

FIG. 9 is a view illustrating a stock registration screen.

FIG. 10 is a view illustrating a wrong rack warning screen.

FIG. 11 is a view illustrating a sterilization validity expiration warning screen.

FIG. 12 is a view illustrating a re-stocking warning screen.

FIG. 13 is a view illustrating a stock registration screen in a state where an instrument set is added to a stock list.

FIG. 14 is a view illustrating a re-stock tabulation screen.

FIG. 15 is a view illustrating a storage history tabulation screen.

### DETAILED DESCRIPTION OF THE PREFERRED EMBODIMENTS

With reference to the attached drawings, storage management systems including storage management devices for medical instruments (each of which will be hereinafter simply referred to as a storage management device) according to preferred embodiments of the present invention will be described below. Note that, as a matter of course, preferred embodiments described herein are not intended to be particularly limiting the present invention.

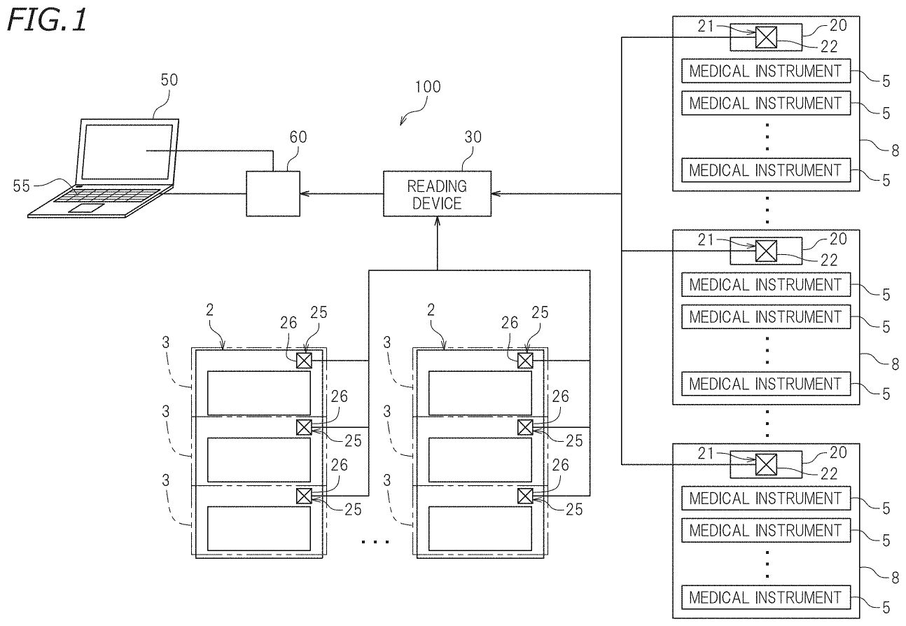

FIG. 1 is a schematic view illustrating a storage management system 100 according to this preferred embodiment. The storage management system 100 is used in storing a medical instrument 5 in a rack number 3 of a predetermined rack 2.

The medical instrument 5 is an instrument that is used in a medical practice that is performed in a medical site, such as a hospital or the like. The medical practice is a practice related to a medical activity that is performed for a patient by a doctor. Examples of a medical practice include a surgical operation and a medical examination.

The medical instrument 5 is, for example, a trocar, forceps, an incision device, a washing and sucking device, scissors, a scalpel (for example, a laser scalpel or an electric scalpel), an ultrasonic cutter, a scalpel holder, a cannula, tweezers, a retractor, a scale, a sonde, an elevator, a rasp, a suction tube, a rib retractor, a rib contractor, a needle holder, an injector, a metal ball, a pus tray, a cup, a pin, a mirror, a file, an opening tool, a clamp, a handpiece, an elevatorium, a chisel, a sharp spoon, a raspatory, a speculum, a suture needle, a punch, a water receiver, a needle, a penetrator, a bougie, a vent pipe, a bone impactor, a luer, a radio plier, a hammer, an angle gauge, a thermometer, a perforator, a spuit, a metal swab, an enema, a syringe, an endoscope, or the like. However, the above-described examples are merely some examples of the medical instrument 5. The medical instrument 5 is not limited to the above-described examples.

The medical instrument 5 may be formed of a single part and may be formed of a plurality of parts. Examples of the medical instrument 5 formed of a plurality of parts include, for example, a trocar, forceps, an incision device, a washing and sucking device, or the like which is used in a laparoscopic surgical operation. The medical instrument 5 described above is formed of a plurality of components, is collected, for example, after a surgical operation, and is disassembled into a plurality of components. Parts that form the medical instrument 5 are referred to as components herein. Also, in some cases, such a component is further formed of a plurality of components, and in such a case, all of the components are referred to as components.

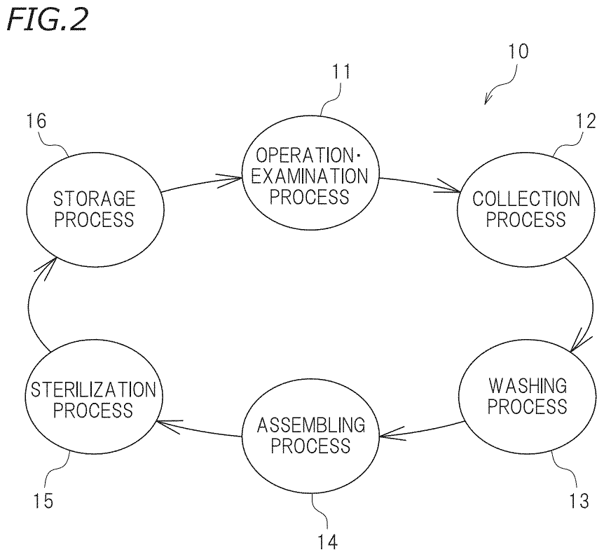

FIG. 2 is an explanatory diagram of a circulation cycle 10. As illustrated in FIG. 2, the medical instrument 5 is repeatedly usable in a predetermined circulation cycle 10. Note that the number of repetitions of use of one medical instrument 5 in the circulation cycle 10 is not particularly limited and is determined in accordance with a type of the medical instrument 5 or a component that forms the medical instrument 5, as appropriate. In this preferred embodiment, the circulation cycle 10 includes an operation and examination process 11, a collection process 12, a washing process 13, an assembling process 14, a sterilization process 15, and a storage process 16.

The operation and examination process 11 is a process in which a surgical operation or a medical examination which is an example of a medical practice is performed using the medical instrument 5. In the operation and examination process 11, the medical instrument 5 that is stored in a rack number 3 of a predetermined rack 2 (see FIG. 1) arranged in a predetermined storage area is dispensed (in other words, is taken out) and a surgical operation or a medical examination is performed using the dispensed medical instrument 5.

The collection process 12 is a process that is performed after the operation and examination process 11. The collection process 12 is a process in which the medical instrument 5 that has been used in a surgical operation or a medical examination is collected. Note that, in a case where the medical instrument 5 is formed of a plurality of components, the collection process 12 may include an operation of disassembling the medical instrument 5 into individual components.

The washing process 13 is a process that is performed after the collection process 12. The washing process 13 is a process in which the medical instrument 5 or each of the individual components of the medical instrument 5 that has been collected in the collection process 12 is washed. In the washing process 13, for example, using a washer (not illustrated), washing of the medical instrument 5 is performed.

The assembling process 14 is a process that is performed after the washing process 13. The assembling process 14 is a process in which the components of the medical instrument 5 which have been washed in the washing process 13 are assembled to complete the single medical instrument 5. The assembling process 14 is a process in which the medical instrument 5 that is formed of a plurality of components is assembled. For example, for the medical instrument 5 that is formed of a single component or the medical instrument 5 that has not been disassembled in the collection process 12, the assembling process 14 may be omitted.

The sterilization process 15 is a process that is performed after the assembling process 14. The sterilization process 15 is a process in which the medical instrument 5 that has been assembled in the assembling process 14 is sterilized. In the sterilization process 15, for example, using a sterilizer (not illustrated), sterilization processing of the medical instrument 5 is performed. Sterilization processing will be hereinafter also referred to merely as sterilization.

The storage process 16 is a process that is performed after the sterilization process 15. The storage process 16 is a process in which the medical instrument 5 on which sterilization processing has been performed is stored in the rack number 3 of the predetermined rack 2 (see FIG. 1) arranged in the predetermined storage area. Herein, storing the medical instrument 5 in the rack number 3 of the rack 2 includes putting the medical instrument 5 in an area of the rack 2 in which the rack number 3 is designated. Storing the medical instrument 5 in the rack number 3 of the predetermined rack 2 will be hereinafter referred to as stocking. Note that, after the storage process 16, the operation and examination process 11 is performed.

In the circulation cycle 10 according to this preferred embodiment, each process is performed using an instrument set 8 illustrated in FIG. 1 as a unit. Herein, the instrument set 8 is a set including one or more medical instruments 5. There is no particular limitation on a method for dividing the plurality of medical instruments 5 into one or more instrument sets 8. The number and types of the medical instruments 5 included in the instrument set 8 are determined for each medical site, such as a hospital or the like and, in some cases, are determined based on empirical rules of staff members, such as a doctor, a nurse, an operator of each process of the circulation cycle 10, or the like.

For example, the instrument set 8 is a set including one or more medical instruments 5 that are used in one medical practice (for example, a surgical operation or a medical examination). The number of instrument sets 8 used in one surgical operation or one medical examination may be one and may be plural. The one or more medical instruments 5 included in one instrument set 8 may be housed in an unillustrated tray in a state where the one or more medical instruments 5 are put and sealed in a predetermined packaging material, and thus, is stored in the storage process 16 (see FIG. 2).

In this preferred embodiment, when sterilization processing is performed on the one or more medical instruments 5 forming the instrument set 8 in the sterilization process 15 illustrated in FIG. 2, a sterilization label 20 illustrated in FIG. 1 is issued. In performing sterilization processing, the one or more medical instruments 5 forming the instrument set 8 are sterilized in a state of being put in the predetermined packaging material. The sterilization label 20 is attached, for example, to the predetermined packaging material described above after sterilization processing. As illustrated in FIG. 1, a sterilization identification mark 21 is assigned to the sterilization label 20. The sterilization identification mark 21 is read by a reading device 30 that will be described later. There is no particular limitation on a type of the sterilization identification mark 21. For example, the sterilization identification mark 21 may be a one-dimensional code, may be a two-dimensional code, and may be an IC tag.

For example, a target sterilization ID 22 is recorded in the sterilization identification mark 21. The target sterilization ID 22 is an ID assigned to the instrument set 8 on which sterilization processing has been performed when the sterilization processing was performed on the instrument set 8. Accordingly, with the target sterilization ID 22 assigned, it can be known that sterilization processing has been performed on the instrument set 8. The target sterilization ID 22 is a specific ID that is assigned to the instrument set 8 each time sterilization processing is performed on the instrument set 8. Therefore, the instrument set 8 can be identified from the target sterilization ID 22. In this preferred embodiment, to one instrument set 8, a new target sterilization ID 22 is assigned each time sterilization processing is performed thereon. The target sterilization ID 22 assigned to the instrument set 8 is changed each time the instrument set 8 is used in a surgical operation or a medical examination and then is sterilized.

In this preferred embodiment, in the storage process 16 of FIG. 2, the one or more medical instruments 5 are stored in units of the instrument set 8. For example, as illustrated in FIG. 1, a plurality of racks 2 are arranged in the storage area where the instrument set 8 is stored. Each of the racks 2 includes a plurality of rack numbers 3. For example, each of the racks 2 is divided into a plurality of areas and a different rack number 3 is assigned to each of the areas. In this preferred embodiment, what type of instrument set 8 is stored in each rack number 3 of each rack 2 is set in advance. However, there is no particular limitation on what type of instrument set 8 is stored in which rack number 3 of which rack 2. What type of instrument set 8 is stored in which rack number 3 of which rack 2 is determined, for example, in each medical site, such as a hospital or the like and, in some cases, is determined based on empirical rules of staff members, such as a doctor, a nurse, an operator of each process of the storage process 16, or the like.

In this preferred embodiment, a rack identification mark 25 is assigned to each rack number 3 of each rack 2. The rack identification mark 25 is read by the reading device 30. There is no particular limitation on a type of the rack identification mark 25. For example, the rack identification mark 25 is read by the reading device 30. There is no particular limitation on the type of the rack identification mark 25 but, for example, the rack identification mark 25 is the same type as that of the sterilization identification mark 21. The rack identification mark 25 may be a one-dimensional code, may be a two-dimensional code, and may be an IC tag.

In the rack identification mark 25, a target rack ID 26 is recorded. The target rack ID 26 is a specific ID assigned to each rack number 3 of each rack 2. The target rack ID 26 assigned to the rack number 3 of the rack 2 is invariable. By reading the target rack ID 26, the rack number 3 of the rack 2 can be identified from the target rack ID 26.

Incidentally, in the operation and examination process 11, for example, when a surgical operation is going to be performed, an operation plan is made. The operation plan includes an instrument list that is a list of the instrument set 8 that is to be used in the surgical operation. An operator takes out the instrument set 8 included in the instrument list from the rack number 3 of the rack 2 in which the instrument set 8 has been stored in the storage process 16 and dispenses the instrument set 8. However, among the instrument sets 8 in the instrument list, that is, the instrument sets 8 that have been dispensed, there can be the instrument set 8 that is not actually used in the surgical operation. In such a case, the instrument set 8 that has not been actually used in the surgical operation is stored again in the storage process 16 without going through the washing process 13, the assembling process 14, and the sterilization process 15 of the circulation cycle 10 illustrated in FIG. 2. That is, in a case where, although the stored instrument set 8 has been dispensed, the instrument set 8 has not been used in the operation and examination process 11, the instrument set 8 is stored again in the storage process 16 without going through sterilization processing or the like. Stocking the instrument set 8 that has not been used in the operation and examination process 11 without performing sterilization processing in the instrument set 8 in the storage process 16 as described above is referred to as “re-stocking.”

Conventionally, re-stocking of the instrument set 8 has not been strictly managed. Depending on a medical site, re-stocking of the instrument set 8 is managed, for example, using recording paper. The re-stocked instrument set 8 has not been used in a surgical operation or a medical examination and has not been essentially needed to be dispensed from the rack number 3 of the rack 2 in which the instrument set 8 was stored. As the number of re-stocked instrument sets 8 increases, the number of works performed for dispensing the instrument sets 8 from the rack numbers 3 of the racks 2 in which the instrument sets 8 are stored increases, and a dispensing work becomes insufficient. Therefore, it is preferable that the number of instrument sets 8 that are re-stocked is not described in the instrument list of the operation plan.

Thus, in this preferred embodiment, the storage management system 100 illustrated in FIG. 1 performs management of stocking of the instrument sets 8 in the storage process 16 (see FIG. 2) and also performs management of re-stocking of the instrument set 8. By using the storage management system 100, for example, the instrument set 8 that is frequently re-stocked is specified and the instrument list of the operation plan is reviewed to increase efficiency of dispensing of the stored instrument sets 8.

In this preferred embodiment, the storage management system 100 includes a reading device 30, a display device 50, an operation device 55, and a storage management device 60.

The reading device 30 is operated by the operator and reads the target sterilization ID 22 by reading the sterilization identification mark 21 of the sterilization label 20 assigned to the instrument set 8. Furthermore, the reading device 30 can read the target rack ID 26 by reading the rack identification mark 25 assigned to the rack number 3 of the rack 2. Although not illustrated, the reading device 30 is provided with a handle that is gripped by the operator's hand.

Note that there is no particular limitation on a type of the reading device 30, and the reading device 30 may be a device that can read the target sterilization ID 22 of the sterilization identification mark 21 and the target rack ID 26 of the rack identification mark 25. For example, in a case where the sterilization identification mark 21 and the rack identification mark 25 are two-dimensional codes, the reading device 30 may be a two-dimensional code reader that can read two-dimensional codes. For example, in a case where the sterilization identification mark 21 and the rack identification mark 25 are IC tags, the reading device 30 may be an IC tag reader. In this preferred embodiment, the reading device 30 is a non-contact type device. However, the reading device 30 may be a contact type device. Note that the number of the reading devices 30 used in the storage process 16 may be one and may be plural. An installation site of the reading device 30 is, for example, the storage area where the rack number 3 of the rack 2 in which the instrument set 8 is stored in the storage process 16 is arranged.

A screen that is used in stocking the instrument set 8 in the storage process 16 is displayed on the display device 50. Note that there is no particular limitation on a type of the display device 50. For example, the display device 50 may be a display of a mobile terminal. The display device 50 may be a display of a desktop type or notebook type (in other words, laptop type) personal computer.

The operation device 55 is used when the operator operates a screen displayed on the display device 50 or the like. The screen displayed on the steering 50 can be switched, for example, by the operator's operation of the operation device 55. Moreover, in which rack number 3 of which rack 2 the instrument set 8 is to be stocked can be registered in the storage management device 60 by the operator's operation of the operation device 55. Note that there is no particular limitation on a type of the operation device 55. For example, the operation device 55 is, for example, a keyboard, a mouse, or the like, of the personal computer. However, the operation device 55 may be a touch panel provided on the display device 50. Note that each of the number of the display devices 50 and the number of the operation devices 55 is one herein, but may be plural.

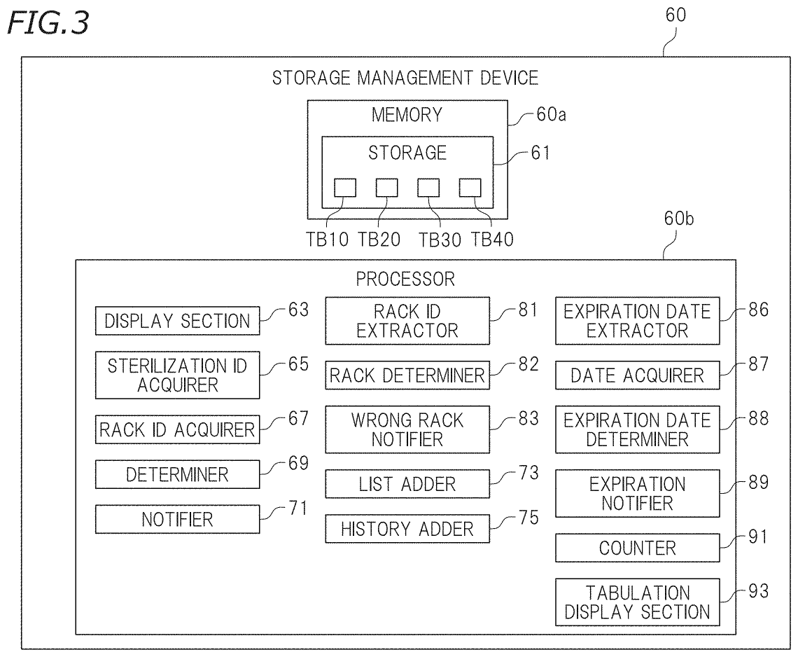

Next, the storage management device 60 according to this preferred embodiment will be described. The storage management device 60 manages storage of the instrument set 8 in the storage process 16 (see FIG. 2), that is, a stock history. Thus, when the instrument set 8 was stocked in the rack number 3 of the predetermined rack 2 can be managed. The storage management device 60 also manages re-stocking of the instrument set 8. The storage management device 60 is a computer control device. FIG. 3 is a block diagram of the storage management device 60. As illustrated in FIG. 3, the storage management device 60 includes a memory 60a and at least one processor 60b. The number of the processors 60b may be one and may be plural. For example, the processor 60b uses a program stored in the memory 60a, and thus, can execute processing. The storage management device 60 may be realized by a dedicated computer and may be realized by a general-purpose computer.

In this preferred embodiment, as illustrated in FIG. 1, the storage management device 60 is communicably connected to the reading device 30. The storage management device 60 may be connected to the reading device 30 via a wired communication and may be connected thereto via a wireless communication. The storage management device 60 is communicably connected to the display device 50 and the operation device 55. Note that the storage management device 60, the display device 50, and the operation device 55 may be realized by one personal computer.

Herein, the storage management system 100 may be realized by a so-called client-server system, and may be realized by a cloud computing. Moreover, the storage management system 100 may be realized by a so-called stand-alone system.

In this preferred embodiment, as illustrated in FIG. 3, the storage management device 60 includes a storage 61, a display section 63, a sterilization ID acquirer 65, a rack ID acquirer 67, a determiner 69, a notifier 71, a list adder 73, and a history adder 75. The storage management device 60 further includes a rack ID extractor 81, a rack determiner 82, a wrong rack notifier 83, an expiration date extractor 86, a date acquirer 87, an expiration determiner 88, and an expiration notifier 89. The storage management device 60 further includes a counter 91 and a tabulation display section 93.

The storage 61 is stored in the memory 60a. The display section 63, the sterilization ID acquirer 65, the rack ID acquirer 67, the determiner 69, the notifier 71, the list adder 73, the history adder 75, the rack ID extractor 81, the rack determiner 82, the wrong rack notifier 83, the expiration date extractor 86, the date acquirer 87, the expiration determiner 88, the expiration notifier 89, the counter 91, and the tabulation display section 93 can be executed by the processor 60b.

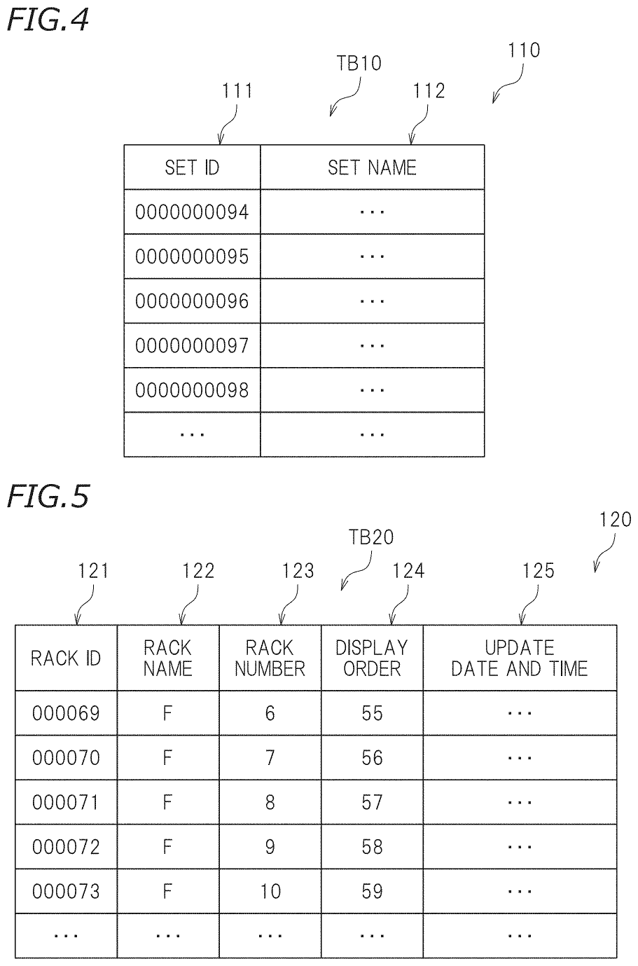

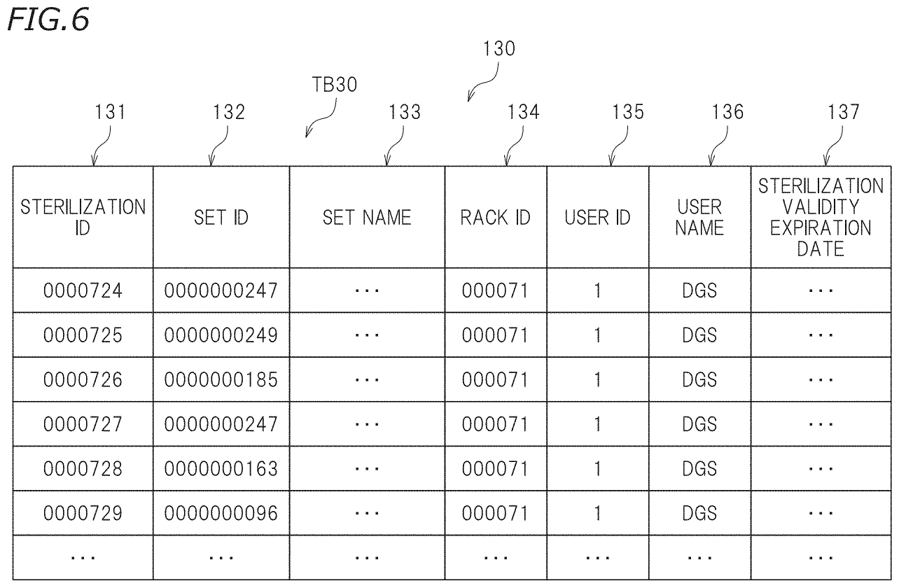

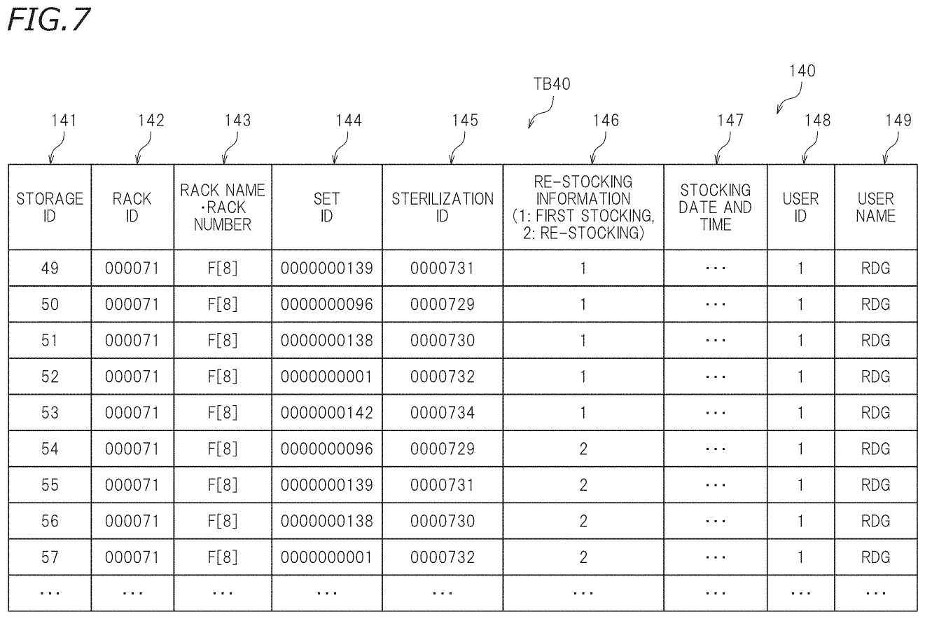

In this preferred embodiment, in the storage 61 realized by the memory 60a, an instrument set master table TB10 (see FIG. 4), a rack master table TB20 (see FIG. 5), a sterilization table TB30 (see FIG. 6), a storage history table TB40 (see FIG. 7) are stored in advance.

As illustrated in FIG. 4, the instrument set master table TB10 is a table in which instrument set information 110 is stored. The instrument set information 110 is, for example, information in which at least a set ID 111 and a set name 112 are associated with one another. In other words, in the instrument set information 110, there are at least items of the set ID 111, the set name 112, and the like.

As used herein, “associated with one another” represents a state where items are described in the same row of the table. “Associated with one another” can be paraphrased as “corresponding to one another.”

As illustrated in FIG. 4, the set ID 111 is a specific ID assigned to each instrument set 8. The set ID 111 is a character string including a number, an alphabet, or the like, and differs for each instrument set 8. The set name 112 is a name of the instrument set 8. The set name 112 may be arbitrarily given to the instrument set 8, for example, by the operator or the like. In this preferred embodiment, the set name 112 is uniquely determined from the set ID 111.

As illustrated in FIG. 5, the rack master table TB20 is a table in which rack information 120 is stored. The rack information 120 is information in which, for example, at least a rack ID 121, a rack name 122, a rack number 123, a display order 124, and an update date and time 125 are associated with one another. Herein, in the rack information 120, there are at least items of the rack ID 121, the rack name 122, the rack number 123, the display order 124, the update date and time 125, and the like.

The rack ID 121 is a specific ID assigned to each rack number 3 of each rack 2 (see FIG. 1) in which the instrument set 8 is stored. The rack ID 121 is a character string including a number, an alphabet, or the like, and differs for each rack number 3 of each rack 2. The rack name 122 is a name of the rack 2. The rack name 122 is arbitrarily given to the rack 2, for example, by the operator or the like. The rack number 123 is a rack number assigned to each rack 2, and is the rack number 3 of the rack 2 (see FIG. 1). In this preferred embodiment, the rack number 3 of the rack 2 indicated in FIG. 1 is specified from the rack ID 121.

The display order 124 is an order in displaying a list of the rack numbers 3 of the racks 2 illustrated in FIG. 1. For example, the list of the rack numbers 3 of the racks 2 is displayed in an ascending order or a descending order of the display order 124. The update date and time 125 is date and time on which the rack information 120 was updated. Herein, when any one of the items of the rack ID 121, the rack name 122, the rack number 123, and the display order 124 associated with the update date and time 125 is changed, the time and date on which the item is changed is added to the update date and time 125.

As illustrated in FIG. 6, the sterilization table TB30 is a table in which sterilization information 130 for the instrument set 8 is stored. The sterilization information 130 is information in which, for example, at least a sterilization ID 131, a set ID 132, a set name 133, a rack ID 134, a user ID 135, a user name 136, and a sterilization validity term expiration date 137 are associated with one another. Herein, in the sterilization information 130, there are at least items of the sterilization ID 131, the set ID 132, the set name 133, the rack ID 134, the user ID 135, the user name 136, the sterilization validity term expiration date 137, and the like.

The sterilization ID 131 is a specific ID assigned to the instrument set 8 on which sterilization processing has been performed in the sterilization process 15 illustrated in FIG. 2. Herein, the instrument set 8 assigned the sterilization ID 131 is determined to have been sterilized. The instrument set 8 can be identified from the sterilization ID 131. The set ID 132 is a set ID assigned to the instrument set 8 assigned the sterilization ID 131. The set ID 132 corresponds to the set ID 111 of the instrument set information 110 (see FIG. 4). The set name 133 illustrated in FIG. 6 is a set name of the instrument set 8 assigned the sterilization ID 131. The set name 133 corresponds to the set name 112 of the instrument set information 110 (see FIG. 4). Note that one of the set ID 132 and the set name 133 may be omitted.

The rack ID 134 illustrated in FIG. 6 is an ID assigned to the rack number 3 of the rack 2 in which the instrument set 8 assigned the sterilization ID 131 is stored. The rack ID 134 corresponds to the rack ID 121 of the rack information 120 (see FIG. 5). The user ID 135 illustrated in FIG. 6 is an ID of the operator who performed sterilization processing on the instrument set 8 assigned the sterilization ID 131. The user name 136 is a name of the operator who performed sterilization processing on the instrument set 8 assigned the sterilization ID 131. Herein, the user ID 135 and the user name 136 correspond to user information (not illustrated) stored in a user master table (not illustrated). The user name 136 can be uniquely determined from the user ID 135. Therefore, one of the user ID 135 and the user name 136 may be omitted.

The sterilization validity term expiration date 137 is an expiration date of a term of validity of sterilization processing that has been performed on the instrument set 8 assigned to the sterilization ID 131. The sterilization validity term expiration date 137 is set to be a date after a predetermined period from issuance of the sterilization label 20 (see FIG. 1). The predetermined period is set based on a type of sterilization processing, a type or types of one or more medical instruments 5 forming the instrument set 8 on which sterilization processing has been performed, or the like.

As illustrated in FIG. 7, the storage history table TB40 is a table in which storage history information 140 that is history information of the instrument set 8 that has been stored in the storage process 16 illustrated in FIG. 2 is stored. The storage history information 140 is information in which, for example, at least a storage ID 141, a rack ID 142, rack name and rack number 143, a set ID 144, a sterilization ID 145, re-stocking information 146, stocking date and time 147, a user ID 148, and a user name 149 are associated with one another. In the storage history information 140, there are at least items of the storage ID 141, the rack ID 142, the rack name and rack number 143, the set ID 144, the sterilization ID 145, the re-stocking information 146, the stocking date and time 147, the user ID 148, the user name 149, and the like.

The storage ID 141 is a specific ID assigned to the stored instrument set 8 when the instrument set 8 is stored in the storage process 16. The storage ID 141 is a character string including a number, an alphabet, or the like, and is issued each time the instrument set 8 is stocked. Therefore, each time one instrument set 8 circulates in the circulation cycle 10 and then is stored in the storage process 16, a different storage ID 141 is issued. Accordingly, a plurality of storage IDs 141 can be assigned to the one instrument set 8.

The rack ID 142 is a rack ID of the rack number 3 of the rack 2 (see FIG. 1) in which the instrument set 8 assigned the storage ID 141 is stored. The rack ID 142 corresponds to the rack ID 121 of the rack information 120 (see FIG. 5). The rack name and rack number 143 illustrated in FIG. 7 is a rack name and a rack number of a rack in which the instrument set 8 assigned the storage ID 141 is stored. Herein, the rack name and rack number 143 are indicated as “RACK NAME [RACK NUMBER].” The rack name and rack number 143 correspond to the rack name 122 (see FIG. 5) and the rack number 123 (see FIG. 5) of the rack information 120. The set ID 144 illustrated in FIG. 7 is a set ID assigned to the instrument set 8 assigned the storage ID 141. The set ID 144 corresponds to the set ID 111 of the instrument set information 110 (see FIG. 4). The sterilization ID 145 illustrated in FIG. 7 is a sterilization ID that is assigned when sterilization processing is performed on the instrument set 8 assigned the storage ID 141. The sterilization ID 145 corresponds to the sterilization ID 131 of the sterilization information 130 (see FIG. 6).

The re-stocking information 146 illustrated in FIG. 7 is information on whether the instrument set 8 assigned the storage ID 141 has been re-stocked. For example, if the associated instrument set 8 is not re-stocked but is stocked for the first time (herein, if stocking of the instrument set 8 is first stocking), “1” is set in the re-stocking information 146. On the other hand, if the associated instrument set 8 has been re-stocked, that is, stocking of the instrument set 8 is a second or subsequent stocking, “2” is set in the re-stocking information 146. However, a specific numerical value or character of the re-stocking information 146 can be set for each medical site as appropriate, the first stocking is not limited to “1,” and the re-stocking is not limited to “2.”

The stocking date and time 147 is a date and a time when the instrument set 8 assigned the storage ID 141 was stocked, that is, a date and a time when the instrument set 8 was stored in the rack number 3 of the rack 2. The user ID 148 is an ID of the operator who stocked the instrument set 8 assigned the storage ID 141. The user name 149 is a name of the operator who stocked the instrument set 8 assigned the storage ID 141. Herein, the user ID 148 and the user name 149 correspond to user information (not illustrated) stored in the above-described user master table (not illustrated). The user name 149 can be uniquely determined from the user ID 148, and therefore, one of the user ID 148 and the user name 149 may be omitted.

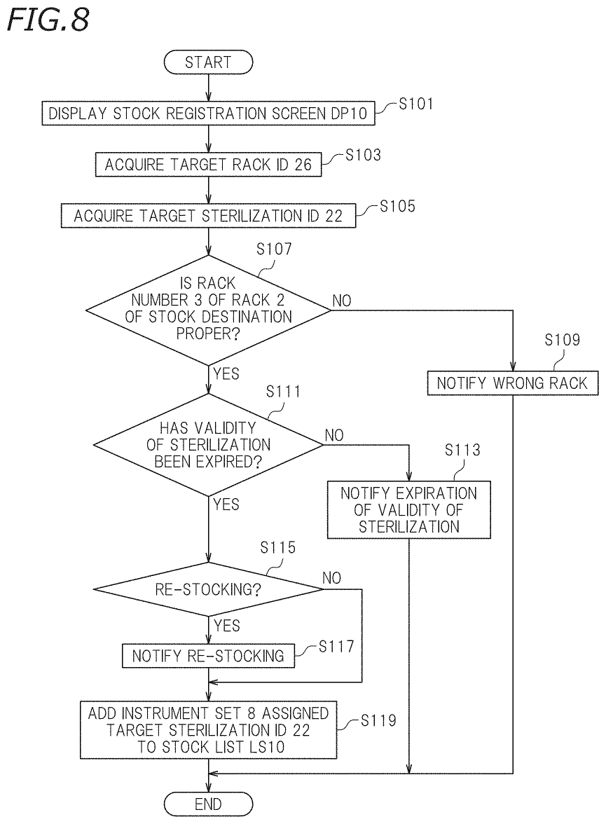

Next, process steps of processing of the storage management device 60 in stocking the instrument set 8 in the storage process 16 of FIG. 2 will be described with reference to a flowchart of FIG. 8.

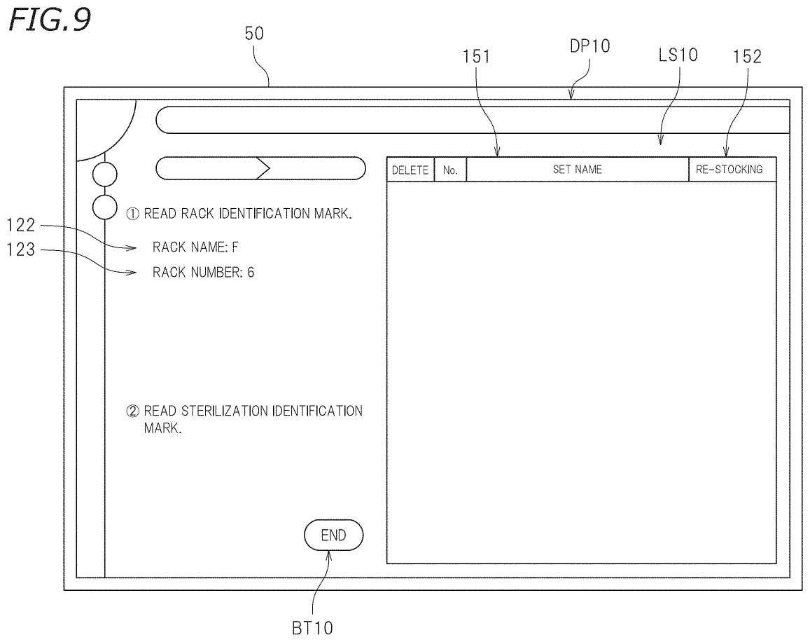

In this preferred embodiment, first, in Step S101, in order to register one or more instrument sets 8 that are to be stocked in a rack number 3 of a predetermined rack 2 (see FIG. 1), the display section 63 of FIG. 3 displays a stock registration screen DP10 illustrated in FIG. 9 on the display device 50, as illustrated in FIG. 9. The stock registration screen DP10 is a screen used in stocking and storing the instrument set 8 in the rack number 3 of the predetermined rack 2 in the storage process 16 and also serves as a screen via which a stocking destination of the instrument set 8 is registered for the storage management device 60.

After the stock registration screen DP10 is displayed on the display device 50 in the above-described manner, in Step S103 of FIG. 8, the rack ID acquirer 67 of FIG. 3 acquires the target rack ID 26 (see FIG. 1) assigned to the rack number 3 of the rack 2 that is the stocking destination of the instrument set 8. Herein, as illustrated in FIG. 1, the target rack ID 26 is read by the reading device 30. The operator holds a reader (not illustrated) of the reading device 30 over the rack identification mark 25 assigned to the rack number 3 of the rack 2 that is a target of stocking in stocking the instrument set 8. Thus, the target rack ID 26 recorded in the rack identification mark 25 can be read by the reading device 30. Accordingly, the rack ID acquirer 67 can acquire the target rack ID 26. Note that the target rack ID 26 acquired by the rack ID acquirer 67 is stored in the storage 61.

In this preferred embodiment, after the target rack ID 26 is acquired, the rack ID 121 that matches the target rack ID 26 is extracted from the rack master table TB20 of FIG. 5 and the rack name 122 and the rack number 123 associated with the extracted rack ID 121 are extracted. Then, as illustrated in FIG. 9, the display section 63 displays the rack name 122 and the rack number 123 that have been extracted on the stock registration screen DP10.

Next, in Step S105 of FIG. 8, the operator registers the instrument set 8 that is to be stocked in the rack number 3 of the rack 2 assigned the target rack ID 26. Herein, the sterilization ID acquirer 65 of FIG. 3 first acquires the target sterilization ID 22 (see FIG. 1) of the instrument set 8 that is a target to be stocked. As illustrated in FIG. 1, the target sterilization ID 22 is read by the reading device 30. The operator holds the reader of the reading device 30 over the sterilization identification mark 21 of the sterilization label 20 attached to the instrument set 8 that is the target to be stocked. Thus, the target sterilization ID 22 recorded in the sterilization identification mark 21 is read by the reading device 30. Accordingly, the sterilization ID acquirer 65 can acquire the target sterilization ID 22. Note that the target sterilization ID 22 acquired by the sterilization ID acquirer 65 is stored in the storage 61.

After the target sterilization ID 22 is acquired by the sterilization ID acquirer 65, in Step S107 of FIG. 8, whether the stocking destination of the instrument set 8 assigned the target sterilization ID 22 is proper is determined. In this preferred embodiment, a stocking destination indicating which rack number 3 of which rack 2 the instrument set 8 is to be stored is set for each instrument set 8 in advance. Herein, as illustrated in FIG. 6, the rack ID 134 of the sterilization information 130 of the sterilization table TB30 is an ID of the rack number 3 of the rack 2 of a proper stocking destination of the instrument set 8 assigned the sterilization ID 131.

Therefore, in this preferred embodiment, in Step S107 of FIG. 8, first, the rack ID extractor 81 of FIG. 3 extracts the rack ID 134 associated with the sterilization ID 131 that matches the target sterilization ID 22 from the sterilization table TB30 illustrated in FIG. 6 and determines the extracted rack ID 134 as an extracted rack ID. Next, the rack determiner 82 of FIG. 3 determines whether the extracted rack ID matches the target rack ID 26 read by the reading device 30 and acquired by the rack ID acquirer 67 in Step S103. If the extracted rack ID matches the target rack ID 26, it is determined that the stocking destination of the instrument set 8 assigned the target sterilization ID 22 is proper. In this case, the process proceeds to Step S111 next.

On the other hand, in Step S107, if it is determined by the rack determiner 82 that the extracted rack ID does not match the target rack ID 26, it is determined that the stocking destination of the instrument set 8 assigned the target sterilization ID 22 is improper and wrong rack is determined. In this case, the process proceeds to Step S109 next. In Step S109, the wrong rack notifier 83 of FIG. 3 notifies the operator of wrong rack. There is no particular limitation on a method for notifying wrong rack.

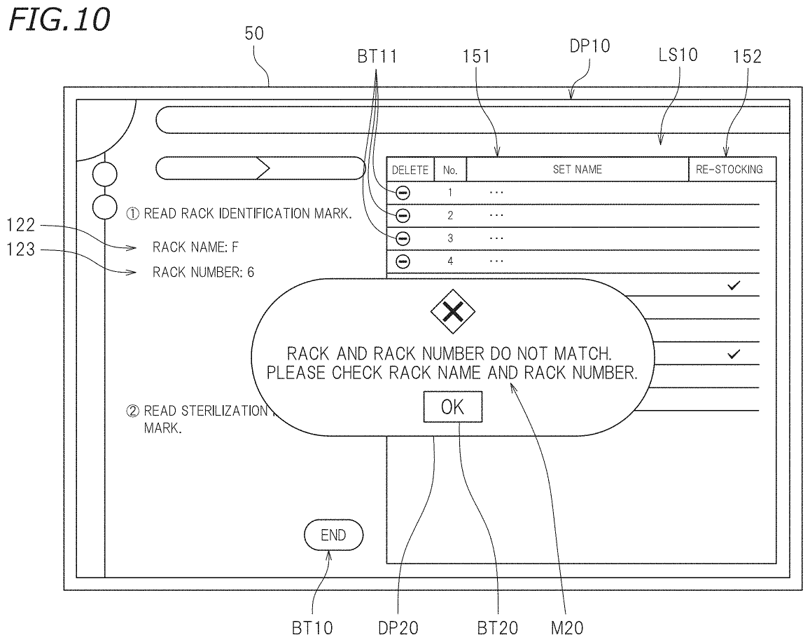

In this preferred embodiment, as illustrated in FIG. 10, the wrong rack notifier 83 displays a wrong rack warning screen DP20 via which a wrong rack is notified on the display device 50. The wrong rack warning screen DP20 is displayed, for example, so as to be superimposed on the stock registration screen DP10. On the wrong rack warning screen DP20, a wrong rack notification message M20 saying, “RACK AND RACK NUMBER DO NOT MATCH. PLEASE CHECK RACK NAME AND RACK NUMBER.” or the like is displayed and also an OK button BT20 is displayed. The operator can know that the rack number 3 of the rack 2 of the stocking destination of the instrument set 8 assigned the target sterilization ID 22 read by the reading device 30 is not proper by viewing the wrong rack notification message M20. Moreover, the operator presses the OK button BT20 via the operation device 55 to close the wrong rack warning screen DP20.

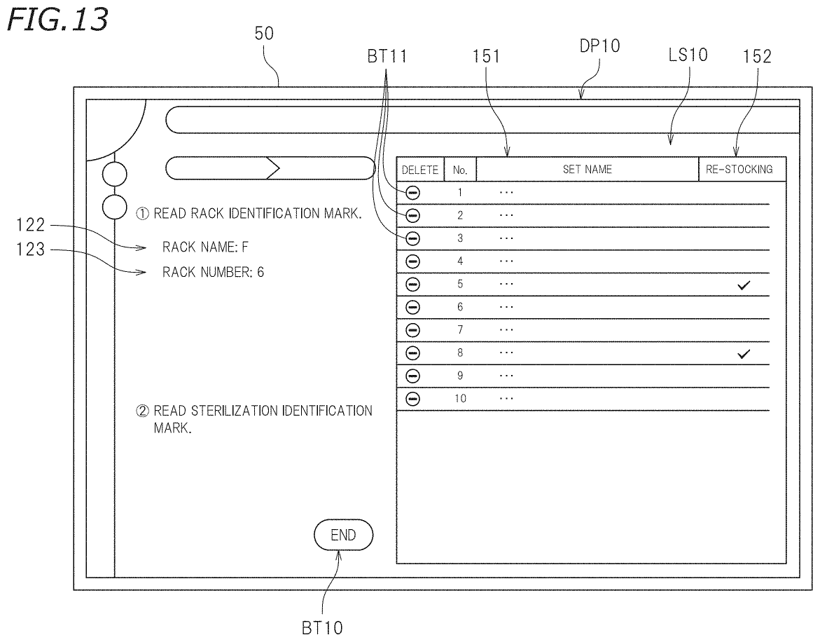

Note that, in this preferred embodiment, in Step S107 of FIG. 8, when a wrong rack is determined, the instrument set 8 assigned the target sterilization ID 22 is not added to a stock list LS10 (see FIG. 13) that will be described later and a flow of process steps of the flowchart of FIG. 8 is terminated.

In this preferred embodiment, after the target sterilization ID 22 is acquired by the sterilization ID acquirer 65, in Step S111 of FIG. 8, whether sterilization processing performed on the instrument set 8 assigned the target sterilization ID 22 is still in a valid period. In this preferred embodiment, as described above, when sterilization processing is performed on the instrument set 8 and the sterilization label 20 (see FIG. 1) is issued, a term of validity of the sterilization processing of the instrument set 8 is set. The term of validity of sterilization processing is set based on a type of sterilization processing or a type or types of one or more medical instruments 5 forming the instrument set 8. In this preferred embodiment, as illustrated in FIG. 6, as the sterilization validity term expiration date 137 of the sterilization information 130 of the sterilization table TB30, a term of validity of sterilization processing for the instrument set 8 assigned the sterilization ID 131 is set.

Therefore, in this preferred embodiment, in Step S111, first, the expiration date extractor 86 of FIG. 3 extracts the sterilization validity term expiration date 137 associated with the sterilization ID 131 that matches the target sterilization ID from the sterilization table TB30 of FIG. 6 and sets the extracted sterilization validity term expiration date 137 as an extracted sterilization validity term expiration date. Next, the date acquirer 87 of FIG. 3 acquires a current date. In this preferred embodiment, in the storage management device 60, date and time information related to current date and time is set. The date acquirer 87 acquires the current date, based on the current date and time information.

Next, the expiration determiner 88 of FIG. 3 determines whether the extracted sterilization validity term expiration date is the same as the current date or after the current date. Herein, if it is determined by the expiration determiner 88 that the extracted sterilization validity term expiration date is the same as the current date or after the current date, it is determined that a term of validity of the sterilization processing for the instrument set 8 assigned the target sterilization ID 22 has not expired. Note that, in this preferred embodiment, determination that a term of validity of sterilization processing has not expired is synonymous with determination that proper sterilization processing has been performed on the instrument set 8 assigned the target sterilization ID 22 and the instrument set 8 has been sterilized. In this case, the process proceeds to Step S115.

On the other hand, in Step S111 of FIG. 8, if it is determined by the expiration determiner 88 that the extracted sterilization validity term expiration date is before the current date, it is determined that the term of validity of the sterilization processing for the instrument set 8 has expired, and thus, expiration of validity of sterilization is determined. In this case, the process proceeds to Step S113 next. In Step S113, the expiration notifier 89 of FIG. 3 notifies the operator of expiration of validity of sterilization. There is no particular limitation on a method for notifying expiration of validity of sterilization.

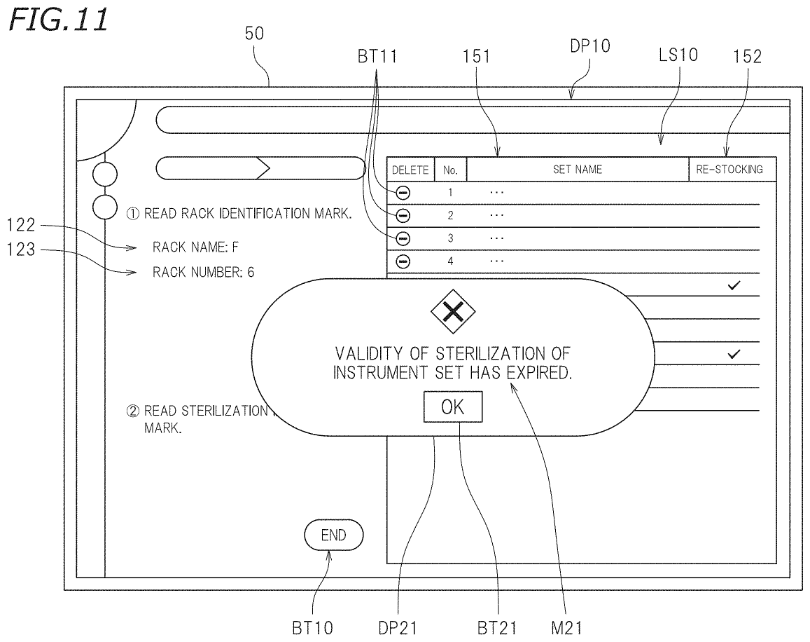

In this preferred embodiment, as illustrated in FIG. 11, the expiration notifier 89 displays a sterilization validity expiration warning screen DP 21 that notifies that validity of sterilization has expired on the display device 50. The sterilization validity expiration warning screen DP 21 is displayed, for example, so as to be superimposed on the stock registration screen DP10. On the sterilization validity expiration warning screen DP 21, a sterilization validity expiration notification message M21 saying, “VALIDITY OF STERILIZATION OF INSTRUMENT SET HAS EXPIRED.” or the like is displayed and also an OK button BT21 is displayed. The operator can know that the term of validity of the sterilization processing for the instrument set 8 assigned the target sterilization ID 22 read by the reading device 30 has expired by viewing the sterilization validity expiration notification message M21. Moreover, the operator presses the OK button BT21 via the operation device 55 to close the sterilization validity expiration warning screen DP 21.

Note that, in this preferred embodiment, in Step S111 of FIG. 8, when expiration of validity of sterilization is determined, the instrument set 8 assigned the target sterilization ID 22 is not added to the stock list LS10 and the flow of process steps of the flowchart of FIG. 8 is terminated. If expiration of validity of sterilization is determined, the process returns to the sterilization process 15 of FIG. 2, sterilization processing is performed on the instrument set 8, and a new sterilization label 20 is issued. Then, after the new sterilization label 20 is issued, the storage process 16 is performed again.

In this preferred embodiment, after the target sterilization ID 22 is acquired by the sterilization ID acquirer 65, in Step S115 of FIG. 8, the determiner 69 of FIG. 3 determines whether the instrument set 8 assigned the target sterilization ID has been re-stocked. In order to determine whether the instrument set 8 has been re-stocked, the determiner 69 determines whether there is the sterilization ID 145 that matches the target sterilization ID 22 in the storage history information 140 (see FIG. 7). Herein, the determiner 69 determines whether there is an ID that matches the target sterilization ID 22 among the sterilization IDs 145 of the storage history information 140 stored in the storage history table TB40 illustrated in FIG. 7.

In this preferred embodiment, if there is the sterilization ID 145 that matches the target sterilization ID 22 in the storage history information 140, it is meant that, after sterilization processing that is to be performed when the sterilization label 20 (see FIG. 1) of the sterilization identification mark 21 in which the target sterilization ID 22 is recorded is issued is performed, the instrument set 8 assigned the target sterilization ID 22 has been already stocked at least once. That is, it is meant that, although the instrument set 8 assigned the target sterilization ID 22 has been dispensed once, the instrument set 8 has not been used in the operation and examination process 11 and has been re-stocked without going through the sterilization processing. As described above, if the sterilization ID 145 that matches the target sterilization ID 22 is included in the storage history information 140, the determiner determines that the instrument set 8 assigned the target sterilization ID 22 has been re-stocked. If it is determined that the instrument set 8 assigned the target sterilization ID 22 has been re-stocked in the above-described manner, the process proceeds to Step S117 of FIG. 8 next.

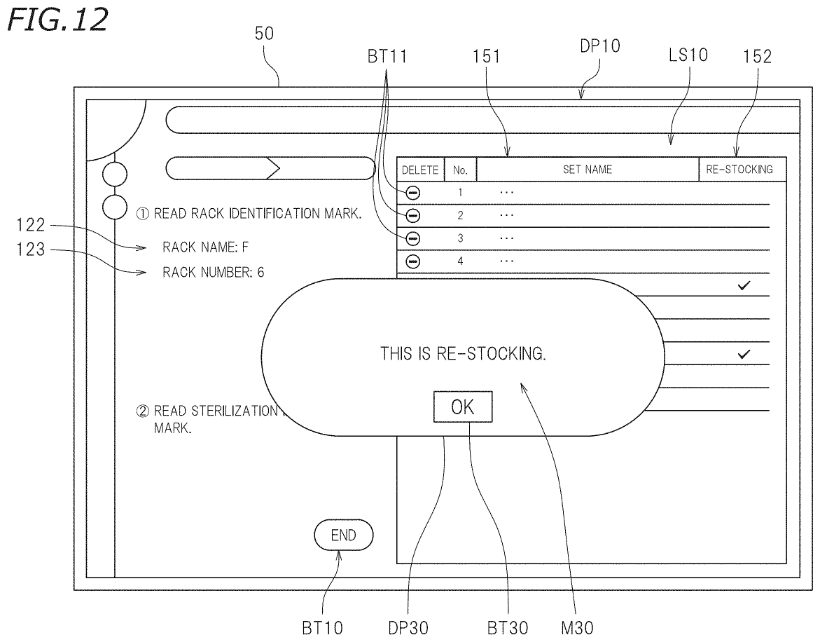

In Step S117, the notifier 71 of FIG. 3 notifies that the instrument set 8 assigned the target sterilization ID 22 has been re-stocked. There is no particular limitation on a method for notifying re-stocking. In this preferred embodiment, as illustrated in FIG. 12, the notifier 71 displays a re-stocking warning screen DP30 that notifies re-stocking on the display device 50. The re-stocking warning screen DP30 is displayed, for example, so as to be superimposed on the stock registration screen DP10. On the re-stocking warning screen DP30, a re-stocking notification message M30 saying, “THIS IS RE-STOCKING.” or the like is displayed and also an OK button BT30 is displayed. The operator can know that the instrument set 8 assigned the target sterilization ID 22 read by the reading device 30 has been re-stocked by viewing the re-stocking notification message M30. Moreover, the operator presses the OK button BT30 via the operation device 55 to close the re-stocking warning screen DP30. In this preferred embodiment, as illustrated in FIG. 8, after Step S117, the process proceeds to Step S119.

On the other hand, in Step S115, if it is determined by the determiner 69 that the sterilization ID 145 that matches the target sterilization ID 22 is not included in the storage history information 140 (see FIG. 7), after the sterilization label 20 (see FIG. 1) of the sterilization identification mark 21 in which the target sterilization ID 22 is recorded is issued, the instrument set 8 assigned the target sterilization ID 22 is stocked for the first time (in other words, stocking of the instrument set 8 is first stocking). That is, after the instrument set 8 assigned the target sterilization ID 22 was used in the operation and examination process 11, sterilization processing has been performed on the instrument set 8 in the sterilization process 15. As described above, if the sterilization ID 145 that matches the target sterilization ID 22 is not included in the storage history information 140, the determiner 69 determines that the instrument set 8 assigned the target sterilization ID 22 is not re-stocked but is stocked for the first time.

Herein, if it is determined by the determiner 69 that the instrument set 8 is not re-stocked, the notifier 71 does not notify re-stocking of the instrument set 8 assigned the target sterilization ID 22. That is, the re-stocking warning screen DP30 illustrated in FIG. 12 is not displayed on the display device 50 and the process proceeds to Step S119 without performing processing of Step S117 of FIG. 8.

In this preferred embodiment, in Step S119, the list adder 73 of FIG. 3 adds the instrument set 8 assigned the target sterilization ID 22 to the stock list LS10 illustrated in FIG. 13. Herein, whether or not the instrument set 8 assigned the target sterilization ID 22 has been re-stocked, the instrument set 8 is added to the stock list LS10. The stock list LS10 is a list displayed on the stock registration screen DP10 by the display section 63, and is a list of the instrument sets 8 that are to be stocked in the rack numbers 3 of the racks 2 assigned the target racks ID 26 acquired via the reading device 30.

The stock list LS10 includes items of a set name 151 and re-stocking information 152. The set name 151 is a set name of the instrument set 8 assigned the target sterilization ID 22. In this preferred embodiment, the sterilization ID 131 that matches the target sterilization ID 22 is extracted from the sterilization information 130 of the sterilization table TB30 illustrated in FIG. 6 and the set name 133 associated with the extracted sterilization ID 131 is extracted. The extracted set name 133 is a set name corresponding to the target sterilization ID 22. Herein, when the instrument set 8 assigned the target sterilization ID 22 is added to the stock list LS10, the list adder 73 adds the extracted set name 133 to the item of the set name 151 of the stock list LS10 displayed on the stock registration screen DP10 to display the extracted set name 133 thereon.

In this preferred embodiment, the re-stocking information 152 of the stock list LS10 is an item indicating whether the instrument set 8 added to the stock list LS10 has been re-stocked. For example, if the instrument set 8 added to the stock list LS10 has been re-stocked, the list adder 73 adds that the instrument set 8 has been re-stocked to the stock list LS10 by entering a check in the re-stocking information 152. In this preferred embodiment, if it is determined that there is the sterilization ID 145 that matches the target sterilization ID 22 in the storage history information 140 (see FIG. 7) and the instrument set 8 assigned the target sterilization ID 22 has been re-stocked, a check is entered in the re-stocking information 152. Note that processing of entering a check in the re-stocking information 152 may be performed by the notifier 71.

In this preferred embodiment, a delete button BT11 corresponding to each row of the stock list LS10 is provided in the stock list LS10. Herein, the operator presses the delete button BT11 via the operation device 55 to delete the instrument set 8 in a row corresponding to the delete button BT11 from the stock list LS10.

In this preferred embodiment, if a plurality of instrument sets 8 are stocked in the rack number 3 of the rack 2 assigned the target rack ID 26, the target sterilization IDs 22 assigned to the plurality of instrument sets 8 can be successively read by the reading device 30. In this case, each time one of the target sterilization IDs 22 assigned to the plurality of instrument sets 8 is read by the reading device 30, processes of Step S105 to Step S119 of FIG. 8 are sequentially executed. Each time one of the target sterilization IDs 22 is read by the reading device 30 and the target sterilization ID 22 is acquired by the sterilization ID acquirer 65, the instrument set 8 assigned the target sterilization ID 22 is added to the stock list LS10.

Herein, after all of the instrument sets 8 that are stocked in the rack number 3 of the rack 2 assigned the target rack ID 26 are added to the stock list LS10, as illustrated in FIG. 13, the operator presses a termination button BT10 displayed on the stock registration screen DP10 via the operation device 55. When the termination button BT10 is pressed, it is determined that the instrument sets 8 added to the stock list LS10 have been stocked in the rack number 3 of the rack 2 assigned the target rack ID 26. Herein, the storage history information 140 related to the instrument sets 8 added to the stock list LS10 is added to the storage history table TB40 illustrated in FIG. 7, so that it is confirmed that the instrument sets 8 have been stocked in the rack number 3 of the rack 2 assigned the target rack ID 26.

When the termination button BT10 is pressed, the history adder 75 of FIG. 3 adds the storage history information 140 related to each instrument set 8 added to the stock list LS10 to the storage history table TB40. Herein, the history adder 75 automatically issues a specific storage ID 141 to each instrument set 8 in the stock list LS10. The history adder 75 adds the target rack ID 26 to the rack ID 142. The history adder 75 extracts the rack name 122 and the rack number 123 associated with the target rack ID 26 from the rack information 120 of the rack master table TB20 (see FIG. 5) and adds the extracted rack name 122 and rack number 123 to the rack name and rack number 143. The history adder 75 extracts the set ID 132 associated with the target sterilization ID 22 from the sterilization information 130 of the sterilization table TB30 (see FIG. 6) and adds the extracted set ID 132 to the set ID 144 of the storage history information 140.

The history adder 75 adds the target sterilization ID 22 to the sterilization ID 145. Information on whether the instrument set 8 assigned the target sterilization ID 22 has been re-stocked is added to the re-stocking information 146. For example, in the stock list LS10, if a check has not been entered in any one of items of the re-stocking information 152, the history adder 75 determines first stocking and adds “1” to the re-stocking information 146. On the other hand, in the stock list LS10, if a check has been entered in any one of the items of the re-stocking information 152, the history adder 75 determines re-stocking, that is, in other words, second or subsequent stocking, and adds “2” to the re-stocking information 146.

The history adder 75 adds a date and a time on which the termination button BT10 was pressed to the stocking date and time 147. The history adder 75 adds the user ID and the user name of the operator who stocked the instrument set 8 to the user ID 148 and the user name 149, respectively. In this preferred embodiment, when the stock registration screen DP10 is displayed on the display device 50, the user ID of the operator is input. The user ID input by the operator at this time is added to the user ID 148 of the storage history information 140, and the user name corresponding to the user ID input by the operator is added to the user name 149 of the storage history information 140.

As described above, after the storage history information 140 related to the instrument set 8 added to the stock list LS10 is added to the storage history table TB40 illustrated in FIG. 7, the operator stocks the instrument set 8 added to the stock list LS10 to the rack number 3 of the rack 2 assigned the target rack ID 26, that is, stores the instrument set 8 in the rack number 3 of the rack 2. In the above-described manner, stocking of the instrument set 8 in the storage process 16 is completed.

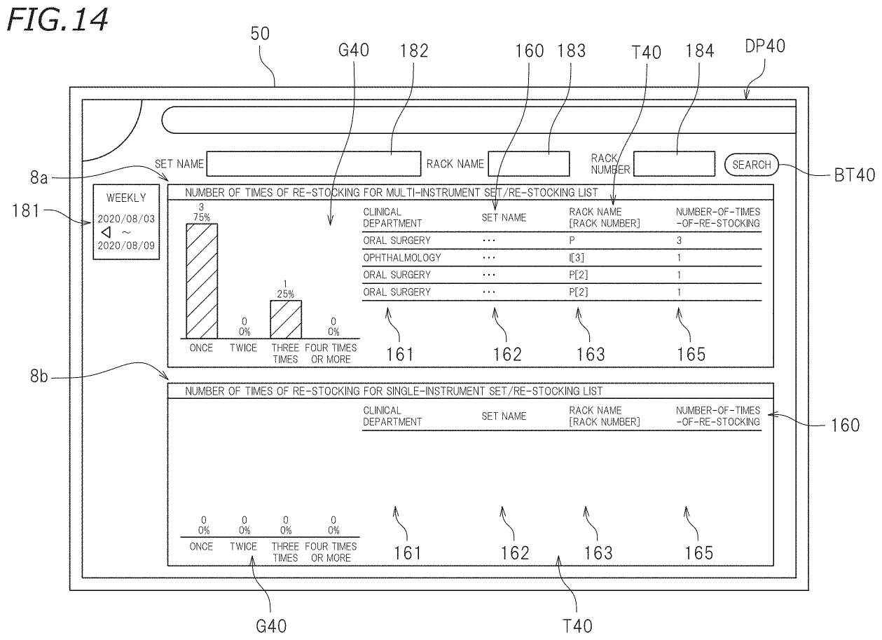

In this preferred embodiment, the tabulation display section 93 of FIG. 3 displays a re-stock tabulation screen DP40 illustrated in FIG. 14 on the display device 50. The re-stock tabulation screen DP40 counts the number of times re-stocking has been performed for each instrument set 8 and displays a result of counting using a graph, a chart, or the like. The operator can know in which instrument set 8 re-stocking is frequently performed by viewing the re-stock tabulation screen DP40.

Herein, the storage management device 60 generates re-stock tabulation information 160 illustrated in FIG. 14. The re-stock tabulation information 160 is generated, for example, based on the storage history information 140 of the storage history table TB40 illustrated in FIG. 7, or the like.

In this preferred embodiment, the counter 91 of FIG. 3 extracts the storage history information 140 in which the re-stocking information 146 indicates re-stocking, that is, the re-stocking information 146 is from the storage history information 140. Next, the counter 91 counts the number of times re-stocking has been performed for each set ID 144. Herein, the counter 91 counts the number of the storage history information 140 that the set ID 144 matches, that is, the number of rows of the storage history table TB40, among the storage history information 140 in which the re-stocking information 146 indicates re-stocking.

In this preferred embodiment, as illustrated in FIG. 14, the re-stock tabulation information 160 is information in which a clinical department 161, a set name 162, rack name and rack number 163, and number-of-times-of-re-stocking 165 are associated with one another. Herein, a set name corresponding to the set ID 144 associated with the re-stocking information 146 indicating re-stocking in the storage history information 140 is added to the set name 162. The set name corresponding to the set ID 144 is acquired, for example, from the instrument set information 110 of the instrument set master table TB10 of FIG. 3. A clinical department in which the instrument set 8 assigned the set ID 144 is used is added to the clinical department 161. In this preferred embodiment, although not illustrated, instrument and clinical department master information in which the set name or the set ID of the instrument set 8 and a clinical department are associated with one another is stored in advance. A clinical department associated with the set name 162 of the re-stock tabulation information 160 is extracted from the instrument and clinical department information and the extracted clinical department is added to the clinical department 161 of the re-stock tabulation information 160.

The rack name and rack number 143 (see FIG. 7) associated with the re-stocking information 146 indicating re-stocking is added to the rack name and rack number 163 of the re-stock tabulation information 160. The number of times re-stocking has been performed for each set ID 144 assigned to the instrument set 8 counted by the counter 91 of FIG. 3 is added to the number-of-times-of-re-stocking 165.

In this preferred embodiment, a re-stock list table T40 and a re-stock graph G40 are arranged and displayed on the re-stock tabulation screen DP40 illustrated in FIG. 14. The re-stock list table T40 is a table in which the instrument set 8 and the number-of-times-of-re-stocking 165 of the instrument set 8 are associated with one another. In the re-stock list table T40, the re-stock tabulation information 160 is displayed and the clinical department 161, the set name 162, the rack name and rack number 163, and the number-of-times-of-re-stocking 165 are associated with one another and displayed.

The re-stock graph G40 is a graph based on the re-stock tabulation information 160 displayed on the re-stock list table T40. In the re-stock graph G40, the number of the instrument sets for each number-of-times-of-re-stocking 165 is graphed and displayed. There is no particular limitation on a type of the graph related to the re-stock graph G40. Herein, the re-stock graph G40 is a bar graph for each number of times of re-stocking. In the re-stock graph G40 in FIG. 14, there is one instrument set 8 for which the number of times of re-stocking is one, and there are three instrument sets 8 for which the number of times of re-stocking is three.

Note that, in this preferred embodiment, the instrument sets 8 can be divided to multi-instrument sets 8a each of which includes a plurality of medical instruments 5 and single-instrument sets 8b each of which is constituted by a single medical instrument 5. Therefore, in the re-stock tabulation screen DP40, the re-stock list table T40 and the re-stock graph G40 may be displayed separately for the multi-instrument sets 8a and the single instrument sets 8b. In one example illustrated in FIG. 14, the re-stock list table T40 and the re-stock graph G40 related to the multi-instrument sets 8a are displayed in an upper portion and the re-stock list table T40 and the re-stock graph G40 related to the single-instrument sets 8b are displayed in a lower portion.

In this preferred embodiment, the re-stock tabulation screen DP40 includes a search function. For example, the re-stock list table T40 and the re-stock graph G40 in accordance with the re-stock tabulation information 160 in which the instrument set 8 and the number-of-times-of-re-stocking are associated with one another, based on a predetermined search period 181, a predetermined search set name 182, or a search rack number 184 of a predetermined search rack 183 that have been designated by the operator may be displayed on the re-stock tabulation screen DP40. Herein, the predetermined search period 181 is, for example, weekly, but may be daily and may be monthly. The search period 181 is configured, for example, by a pull-down menu. Each of the search set name 182, the search rack 183, and the search rack number 184 may be configured by a text box input by the operator and may be configured by a pull-down menu.

In this preferred embodiment, a search button BT40 is arranged in the re-stock tabulation screen DP40. The operator presses the search button BT40 via the operation device 55, so that the tabulation display section 93 displays the re-stock list table T40 and the re-stock graph G40 based on conditions designated by the search period 181, the search set name 182, and the search rack number 184 of the search rack 183 on the re-stock tabulation screen DP40.

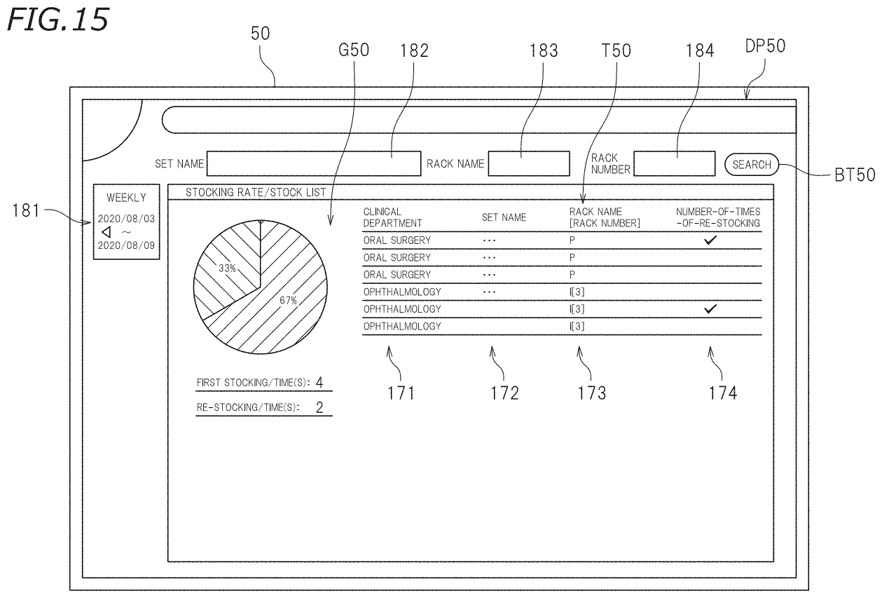

In this preferred embodiment, the tabulation display section 93 of FIG. 3 can display a storage history tabulation screen DP50 illustrated in FIG. 15 on the display device 50. A storage history list table T50 and a stock rate graph G50 are displayed on the storage history tabulation screen DP50.

The storage history list table T50 is a table generated based on the storage history information 140 of the storage history table TB40 of FIG. 7. In the storage history list table T50, regardless of re-stocking or not, the instrument set 8 that satisfies predetermined conditions (for example, a predetermined period) is displayed. The storage history list table T50 is information in which a clinical department 171, a set name 172, a rack name and a rack number 173, and a re-stocking information 174 are associated with one another. The clinical department 171 is acquired by similar procedures to those used for acquiring the clinical department 161 of the re-stock tabulation information 160 of FIG. 14. The set name 172, the rack name and rack number 173, and the re-stocking information 174 are acquired from a set name corresponding to the set ID 144 of the storage history table TB40 of FIG. 7, the rack name and rack number 143, and the re-stocking information 146. In the re-stocking information 174, in a case of re-stocking, that is, if the re-stocking information 146 is “2,” a check is entered.

The stock rate graph G50 is a graph generated based on the storage history list table T50. The stock rate graph G50 is a pie chart illustrating, for the instrument sets 8 displayed on the storage history list table T50, a ratio of the number of times (cumulative number of times) of first stocking to the total number of times (cumulative number of times) all of the instrument sets 8 in the storage history list table T50 have been stocked and a ratio of the number of time (cumulative number of times) of re-stocking to the total number of times (cumulative number of times) all of the instrument sets 8 in the storage history list table T50 have been stocked. Thus, based on the predetermined conditions, the ratio of the number of times of first stockings to the total number of times all of the instrument sets 8 have been stocked can be known. Moreover, based on the predetermined conditions, the ratio of the number of times of re-stocking to the total number of times all of the instrument sets 8 have been stocked can be known.

Note that the storage history tabulation screen DP50 illustrated in FIG. 15 also has a similar search function to that of the re-stock tabulation screen DP40 illustrated in FIG. 14. Herein, the operator presses a search button BT50 via the operation device 55 to display the storage history list table T50 and the stock rate graph G50 based on the conditions designated by the search period 181, the search set name 182, and the search rack number 184 of the search rack 183.

As described above, in this preferred embodiment, as illustrated in FIG. 3, the storage management device 60 for the medical instrument 5 includes the memory 60a and the at least one processor 60b. The memory 60a stores the storage history information 140. As illustrated in FIG. 7, the storage history information 140 is information in which at least the storage ID 141 that is assigned to the instrument set 8 when the instrument set 8 including one or more medical instruments 5 is stored and the sterilization ID 145 indicating that sterilization processing has been performed on the instrument set 8 are associated with one another. The at least one processor 60b (see FIG. 3) is configured or programmed to execute sterilization ID acquisition processing, determination processing, and notification processing. The sterilization ID acquisition processing is executed by the sterilization ID acquirer 65 of FIG. 3. In the sterilization ID acquisition processing, as in Step S105 of FIG. 8, the target sterilization ID 22 assigned to the instrument set 8 that is a target to be stocked is acquired. The determination processing is executed by the determiner 69 of FIG. 3. In the determination processing, as in Step S115 of FIG. 8, whether there is the sterilization ID 145 that matches the target sterilization ID 22 in the storage history information 140 (see FIG. 7) is determined. The notification processing is executed by the notifier 71 of FIG. 3. In the notification processing, as in Step S117 of FIG. 8, when it is determined in the determination processing that there is the sterilization ID 145 that matches the target sterilization ID 22 in the storage history information 140, it is notified that the instrument set 8 assigned the target sterilization ID 22 has been re-stocked.

In this preferred embodiment, the target sterilization ID 22 illustrated in FIG. 1 is an ID that is assigned when sterilization processing is performed on the instrument set 8 in the sterilization process 15 (see FIG. 2). The sterilization processing is performed on the instrument set 8 that has been used in a surgical operation, a medical examination, or the like in the operation and examination process 11 and, even to the same instrument set 8, each time the sterilization processing is performed, a new target sterilization ID 22 is assigned. Herein, if there is the sterilization ID 145 that matches the target sterilization ID 22 acquired in the sterilization ID acquisition processing in the storage history information 140, it is meant that the instrument set 8 that has been dispensed once is stocked again without being used in a surgical operation or a medical examination and without going through sterilization processing. Accordingly, when it is determined that there is the sterilization ID 145 that matches the target sterilization ID 22 in the storage history information 140, it can be determined that the instrument set 8 assigned the target sterilization ID 22 has been re-stocked. Then, in the notification processing, it is notified that the instrument set 8 assigned the target sterilization ID 22 has been re-stocked, so that the operator can be easily know the instrument set 8 that has been re-stocked.

In this preferred embodiment, in the notification processing, the notifier 71 of FIG. 3 displays the re-stocking warning screen DP30 including the re-stocking notification message M30 indicating that the instrument set 8 assigned the target sterilization ID 22 has been re-stocked (see FIG. 12) on the display device 50. Thus, as illustrated in FIG. 12, the operator can know that the instrument set 8 assigned the target sterilization ID 22 has been re-stocked by viewing the re-stocking warning screen DP30 on the display device 50. Accordingly, the operator can easily visually know that the instrument set 8 assigned the target sterilization ID 22 has been re-stocked.

In this preferred embodiment, as illustrated in FIG. 7, the memory 60a of FIG. 3 stores the storage history table TB40 in which the storage history information 140 is stored. Thus, the storage history information 140 is managed by a table of a database, and therefore, addition, deletion, and change of the storage history information 140 can be easily performed.

In this preferred embodiment, as illustrated in FIG. 7, in the storage history information 140, the sterilization ID 145, the rack ID 142 indicating the rack number 3 of the rack 2 in which the instrument set 8 is stored, and the re-stocking information 146 indicating whether the instrument set 8 has been re-stocked are further associated. The at least one processor 60b is configured or programmed to execute rack ID acquisition processing and history addition processing. The rack ID acquisition processing is executed by the rack ID acquirer 67 of FIG. 3. In the rack ID acquisition processing, as in Step S103 of FIG. 8, the target rack ID 26 assigned to the rack number 3 of the rack 2 in which the instrument set 8 is stored is acquired. The history addition processing is executed by the history adder 75 of FIG. 3. In the history addition processing, a specific ID that has been automatically assigned is determined as the storage ID 141, the target sterilization ID 22 acquired by the sterilization ID acquisition processing is determined as the sterilization ID 145, the target rack ID 26 acquired by the rack ID acquisition processing is determined as the rack ID 142, and, when it is determined in the determination processing that the sterilization ID 145 that matches the target sterilization ID 22 is included in the storage history information 140, the storage history information 140 in which re-stocking is indicated as the re-stocking information 146 is generated and is added to the storage history table TB40 illustrated in FIG. 7. In the history addition processing, when it is determined in the determination processing that the sterilization ID 145 that matches the target sterilization ID 22 is not included in the storage history information 140, no re-stocking (herein, first stocking) is determined as the re-stocking information 146.

Thus, as illustrated in FIG. 7, in the storage history information 140, the sterilization ID 145 and the re-stocking information 146 are associated with one another. The storage history information 140 including the re-stocking information 146 is generated and added to the storage history table TB40 at a timing at which the target rack ID 26 is acquired by the rack ID acquisition processing and the target sterilization ID 22 is acquired by the sterilization ID acquisition processing. The operator can easily know which instrument set 8 assigned which sterilization ID 145 has been re-stocked by checking the re-stocking information 146 of the storage history information 140.

In this preferred embodiment, as illustrated in FIG. 7, in the storage history information 140, the sterilization ID 145 and the set ID 144 used for determining the instrument set 8 are further associated. The at least one processor 60b is configured or programmed to execute counting processing and tabulation display section processing. The counting processing is executed by the counter 91 of FIG. 3. In the counting processing, for each set ID 144, the number-of-times-of-re-stocking 165 (see FIG. 14) is calculated by counting the storage history information 140 in which the re-stocking information 146 indicates re-stocking. The tabulation display section processing is executed by the tabulation display section 93 of FIG. 3. In the tabulation display section processing, for each set ID 144, the re-stock tabulation screen DP40 on which the number-of-times-of-re-stocking 165 for the instrument set 8 assigned the set ID 144 calculated by the counting processing is indicated (see FIG. 14) is displayed on the display device 50. Thus, the operator can know the instrument set 8 assigned the set ID 144 that is frequently re-stocked by viewing the re-stock tabulation screen DP40 displayed on the display device 50. Accordingly, the number of re-stockings of the instrument set can be reduced by reexamining the instrument sets in the operation plan including the instrument set 8 that is frequently re-stocked. As a result, the stored instrument sets 8 can be efficiently dispensed.

In this preferred embodiment, as illustrated in FIG. 1, the storage management system 100 includes the storage management device 60 for the medical instrument 5, the display device 50, the operation device 55, and the reading device 30. The sterilization identification mark 21 in which the target sterilization ID 22 is recorded is assigned to the instrument set 8. The reading device is operated by the operator and can read the sterilization identification mark 21. In the sterilization ID acquisition processing of the storage management device 60, the target sterilization ID 22 is acquired by reading the sterilization identification mark 21 by the reading device 30. Thus, the operator uses a simple method in which the sterilization identification mark 21 of the instrument set 8 that is a target to be stocked is read by the reading device 30, so that the storage management device 60 can acquire the target sterilization ID 22.

In this preferred embodiment, as illustrated in FIG. 1, the rack identification mark 25 in which the target rack ID 26 is recorded is assigned to the rack number 3 of the predetermined rack 2 in which the instrument set 8 is stored. The reading device 30 is configured to read the rack identification mark 25. The at least one processor 60b of the storage management device 60 is configured or programmed to execute rack ID acquisition processing, display processing, and list addition processing. In the rack ID acquisition processing, as in Step S103 of FIG. 8, the rack ID acquirer 67 of FIG. 3 acquires the target rack ID 26 by reading the rack identification mark 25 by the reading device 30. The display processing is executed by the display section 63 of FIG. 3. In the display processing, as in Step S101 of FIG. 8, the stock registration screen DP10 (see FIG. 9) on which the stock list LS10 that is a list of the instrument sets 8 that are to be stocked in the target rack ID 26 acquired by the rack ID acquisition processing is displayed on the display device 50. The list addition processing is executed by the list adder 73 of FIG. 3. In the list addition processing, as in Step S119 of FIG. 8, in the sterilization ID acquisition processing, when the target sterilization ID 22 is acquired, the instrument set 8 assigned the target sterilization ID 22 is add to the stock list LS10 (see FIG. 13). Furthermore, in the list addition processing, when it is determined in the determination processing that there is the sterilization ID 145 that matches the target sterilization ID 22 in the storage history information 140, it is added to the stock list LS10 that the instrument set 8 assigned the target sterilization ID 22 has been re-stocked. Herein, as illustrated in FIG. 13, it is added to the stock list LS10 that the instrument set 8 assigned the target sterilization ID 22 has been re-stocked by entering a check in the re-stocking information 152 of the stock list LS10.

Thus, information indicating re-stocking is added to the stock list LS10 of the instrument set 8 that is stocked in the rack number 3 of the rack 2 assigned the target rack ID 26, so that the operator can know at a glance that, among the instrument sets 8 that are to be stocked, which instrument set 8 has been re-stocked by viewing the stock list LS10.

In this preferred embodiment, the storage management method for the medical instrument 5 can be realized by the storage management device 60 for the medical instrument 5. The storage management method for the medical instrument 5 according to this preferred embodiment includes a display step, a sterilization ID acquisition step, a rack ID acquisition step, a determination step, a notification step, a list addition step, a history addition step, a rack ID extract step, a rack ID determination step, a wrong rack notification step, an expiration date extraction step, a date acquisition step, an expiration determination step, an expiration notification step, a counting step, and a tabulation display section step. The display step, the sterilization ID acquisition step, the rack ID acquisition step, the determination step, the notification step, the list addition step, the history addition step, the rack ID extract step, the rack ID determination step, the wrong rack notification step, the expiration date extraction step, the date acquisition step, the expiration determination step, the expiration notification step, the counting step, and the tabulation display section step are realized by the display section 63, the sterilization ID acquirer 65, the rack ID acquirer 67, the determiner 69, the notifier 71, the list adder 73, the history adder 75, the rack ID extractor 81, the rack determiner 82, the wrong rack notifier 83, the expiration date extractor 86, the date acquirer 87, the expiration determiner 88, the expiration notifier 89, the counter 91, and the tabulation display section 93 of the storage management device 60, respectively.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A storage management device for a medical instrument, the storage management device comprising:
a memory; and
at least one processor; wherein
storage history information in which at least a storage ID that is assigned to an instrument set when the instrument set including one or more medical instruments is stored in the memory and a sterilization ID indicating that sterilization processing has been performed on the instrument set are associated with one another is stored in the memory; and
the at least one processor is configured or programmed to execute a program stored in the memory to perform:
sterilization ID acquisition processing to acquire a target sterilization ID assigned to an instrument set that is a target to be stocked;
determination processing to determine whether the sterilization ID that matches the target sterilization ID is included in the storage history information; and
notification processing to notify, when it is determined in the determination processing that the sterilization ID that matches the target sterilization ID is included in the storage history information, that the instrument set assigned the target sterilization ID has been re-stocked.

2. The storage management device for a medical instrument according to claim 1, wherein, in the notification processing, a re-stocking warning screen including a re-stocking notification message indicating that the instrument set assigned the target sterilization ID has been re-stocked is displayed on a display device.

3. The storage management device for a medical instrument according to claim 1, wherein a storage history table including the storage history information is stored in the memory.

4. The storage management device for a medical instrument according to claim 3, wherein
in the storage history information, the sterilization ID, a rack ID indicating a rack number of a rack in which the instrument set is stored, and re-stocking information indicating whether the instrument set has been re-stocked are further associated; and
the at least one processor is configured or programmed to execute the program stored in the memory to perform:
rack ID acquisition processing to acquire a target rack ID assigned to a rack number of a predetermined rack in which the instrument set is stored; and
history addition processing to determine a specific ID that has been automatically assigned as the storage ID, the target sterilization ID acquired by the sterilization ID acquisition processing as the sterilization ID, and the target rack ID acquired by the rack acquisition processing as the rack ID, generate, when it is determined in the determination processing that the sterilization ID that matches the target sterilization ID is included in the storage history information, the storage history information in which re-stocking is indicated as the re-stocking information, and add the generated stocking history information to the storage history table.

5. The storage management device for a medical instrument according to claim 4, wherein, in the history addition processing, when it is determined in the determination processing that the sterilization ID that matches the target sterilization ID is not included in the storage history information, no re-stocking is indicated as the re-stocking information.

6. The storage management device for a medical instrument according to claim 1, wherein
in the storage history information, the sterilization ID and a set ID used to determine the instrument set are further associated; and
the at least one processor is configured or programmed to execute the program stored in the memory to perform:
counting processing to calculate, for each set ID, a number of times of re-stocking by counting the storage history information in which the re-stocking information is the re-stocking; and
tabulation display section processing to display, for each set ID, a re-stocking tabulation screen on which the number of times of re-stocking for the instrument set assigned the set ID calculated by the counting processing is indicated on the display device.

7. A storage management system comprising:
the storage management device for a medical instrument according to claim 1;
a display device; and
an operation device.

8. The storage management system according to claim 7, further comprising:
a reading device that is operated by an operator to read a sterilization identification mark; wherein
in the sterilization identification mark, the target sterilization ID is recorded, and the sterilization identification mark is assigned to the instrument set; and
in the sterilization ID acquisition processing, the target sterilization ID is acquired by the reading device reading the sterilization identification mark.

9. The storage management system according to claim 8, wherein
a rack identification mark in which a target rack ID is recorded is assigned to a rack number of a predetermined rack in which the instrument set is stored;
the reading device is configured to read the rack identification mark; and
the at least one processor is configured or programmed to execute the program stored in the memory to perform:
rack ID acquisition processing to acquire the target rack ID by the reading device reading the rack identification mark;
display processing to display a stock registration screen on which a stock list that is a list of the instrument set that is to be stocked in the target rack ID acquired by the rack ID acquisition processing is displayed; and
list addition processing to add, when the target sterilization ID is acquired in the sterilization ID acquisition processing, the instrument set assigned the target sterilization ID to the stock list and add to the stock list, when it is determined in the determination processing that the sterilization ID that matches the target sterilization ID is included in the storage history information, that the instrument set assigned the target sterilization ID has been re-stocked.

10. A storage management method for a medical instrument, the storage management method comprising:
storing storage history information in which at least a storage ID that is assigned to an instrument set including one or more medical instruments when the instrument set is stored and a sterilization ID indicating that sterilization processing has been performed on the instrument set are associated with one another;
a sterilization ID acquisition step of acquiring a target sterilization ID assigned to an instrument set that is a target to be stocked;
a determination step of determining whether the sterilization ID that matches the target sterilization ID is included in the storage history information; and
a notification step of notifying, when it is determined in the determination step that the sterilization ID that matches the target sterilization ID is included in the storage history information, that the instrument set assigned the target sterilization ID has been re-stocked.

11. The storage management method for a medical instrument according to claim 10, wherein
a storage history table in which the storage history information is stored; and
in the storage history information, the sterilization ID, a rack ID indicating a rack number of a rack in which the instrument set is stored, and re-stocking information indicating whether the instrument set has been re-stocked are further associated; and
the storage management method further includes:
a rack ID acquisition step of acquiring a target rack ID assigned to a rack number of a predetermined rack in which the instrument set is stored; and
a history addition step of determining a specific ID that has been automatically assigned as the storage ID, the target sterilization ID acquired by the sterilization ID acquisition step as the sterilization ID, and the target rack ID acquired by the rack acquisition step as the rack ID, generating, when it is determined in the determination step that the sterilization ID that matches the target sterilization ID is included in the storage history information, the storage history information in which re-stocking is indicated as the re-stocking information, and adding the generated stocking history information to the storage history table.

12. The storage management method for a medical instrument according to claim 11, wherein, in the history addition step, when it is determined in the determination step that the sterilization ID that matches the target sterilization ID is not included in the storage history information, no re-stocking is indicated as the re-stocking information.

13. The storage management method for a medical instrument according to claim 10, wherein
in the storage history information, the sterilization ID and a set ID used to determine the instrument set are further associated; and
the storage management method further includes:
a counting step of calculating, for each set ID, a number of times of re-stocking by counting the storage history information in which the re-stocking information is the re-stocking; and
a tabulation display step of displaying, for each set ID, a re-stocking tabulation screen on which the number of times of re-stocking for the instrument set assigned the set ID calculated by the counting step is indicated on a display device.

14. The storage management method for a medical instrument according to claim 10, wherein
a sterilization identification mark in which the target sterilization ID is recorded is assigned to the instrument set; and
in the sterilization ID acquisition step, the target sterilization ID is acquired by a reading device reading the sterilization identification mark.

15. The storage management method for a medical instrument according to claim 14, wherein
a rack identification mark in which a target rack ID is recorded is assigned to a rack number of a predetermined rack in which the instrument set is to be stored; and
the storage management method further includes:
a rack ID acquisition step of acquiring the target rack ID by reading the rack identification mark by the reading device;
a display step of displaying on a display device a stock registration screen on which a stock list that is a list of the instrument set that is to be stocked in the target rack ID acquired by the rack ID acquisition step; and
a list addition step of adding, when the target sterilization ID is acquired in the sterilization ID acquisition step, the instrument set assigned the target sterilization ID to the stock list and adding to the stock list, when it is determined in the determination step that the sterilization ID that matches the target sterilization ID is included in the storage history information, that the instrument set assigned the target sterilization ID has been re-stocked.
